(19) United States
(12) Patent Application Publication
(10) Pub. No.: US 2015/0265669 A1
(43) Pub. Date: Sep. 24, 2015

(54) METHODS AND COMPOSITIONS FOR INHIBITING CANCER CELL GROWTH

(71) Applicant: Universiti Putra Malaysia, Selangor (MY)

(72) Inventors: Suhaila Mohamed, Selangor (MY); Negin Ahmadi, Selangor (MY); Swee Ling Lim, Selangor (MY); Noordin M Mustapha, Selangor (MY); Heshu Sulaiman Rahman, Selangor (MY)

(21) Appl. No.: 14/661,751

(22) Filed: Mar. 18, 2015

(30) Foreign Application Priority Data
Mar. 20, 2014 (MY) PI 2014700663

(51) Int. Cl. A61K 36/9068; A61K 36/746; A61K 36/19; A61K 36/74

(52) U.S. Cl. CPC A61K 36/9068; A61K 36/74; A61K 36/746; A61K 36/19

(57) ABSTRACT

The present invention discloses compositions and methods for treating, as well as preventing and inhibiting blood and lung cancer by providing a safe, nutraceutical composition comprising a plant extract from the family Rubiaceae, Acanthaceae and Zingiberaceae. In more specific, the nutraceutical composition is capable of inducing apoptosis in leukemia and lung cancer cells, to reduce severity and incidence, without affecting growth of normal cells.

### CROSS-REFERENCE TO RELATED APPLICATIONS

This application claims the benefit of Malaysian Application No. PI 2014700663, filed on Mar. 20, 2014, the content of which is hereby incorporated by reference in its entirety.

### BACKGROUND OF THE INVENTION

1. Field of the Invention

The present invention relates to methods and compositions to inhibit, block, and prevent further growth of carcinogenic cells, as well as to destroy existing carcinogenic cells. More particularly, the present invention relates to composition comprising an extract of a plant from the family Rubiaceae, Acanthaceae and Zingiberaceae for inhibiting, blocking, and preventative methods of treatment for cancer, including blood and lung cancer.

2. Background Information

Cancer and its treatment is tissue specific. Leukemia is a blood or bone marrow cancer characterized by an abnormal increase of immature white blood cells called “blasts”, and is a kind of hematological neoplasms diseases affecting the blood, bone marrow, and lymphoid system. Leukemia is usually treated with chemotherapy, medical radiation therapy, hormone treatments, or bone marrow transplant. The rate of recovery depends on the type of leukemia and patients' age, the younger the better. Most chronic and acute leukemia cases can be successfully managed for years. Blastic leukemia, involves the same treatment, but is usually less successful. In 2000 about 256,000 children and adults worldwide developed leukemia and 209,000 died from it. About 90% of all leukemia is diagnosed in adults. Leukemia is a malignant hematopoietic disease characterized by an uncontrolled proliferation and block in differentiation of hematopoietic cells. The severity may or may not be linked to impaired immune functions.

Lung cancer is an uncontrolled lung tissues cell growth, which can spread or metastasise beyond the lung into nearby or other tissues of the body. Most primary lung cancers, originating in the lung, are carcinomas from epithelial cells. Lung cancer is subcategorized as small-cell lung carcinoma (SCLC), or oat cell cancer, and non-small-cell lung carcinoma (NSCLC). Symptoms include coughing (sometimes with blood), weight loss and shortness of breath. Lung cancer are 80-90% caused by long-temm exposure to tobacco smoke and 10-15% are attributed to a genetic factors, radon gas, asbestos, and air pollution or second-hand smoke. Treatment and long-term outcomes depend on the type of cancer, the stage (degree of spread), and the person's overall health. Common treatments are surgery, chemotherapy, and radiotherapy. NSCLC is sometimes treated with surgery, whereas SCLC usually responds better to chemotherapy and radiotherapy. About 15% of people diagnosed with lung cancer survive five years after the diagnosis. Worldwide, lung cancer is the most common cause of cancer-related death in men and women, and is responsible for 1.38 million deaths annually (2008 statistics).

Morinda citrifolia fruit is believed to have broad therapeutic effects including anti-cancer and growth prohibiting activity. Morinda citrifolia, as processed and administered according to the specification of United States publication number US20040086583, possesses an anti-angiogenic solution, or rather possesses anti-angiogenic effects, when prophylactically administered and applied to the tumorous regions within the body. In this publication, Morinda citrifolia fruit effectively inhibits and prevents the generation and formation of additional blood vessels within tumors, thus inhibiting and preventing further growth of the tumor, to completely inhibit angiogenesis in the tubule elongation and the endothelial cell migration phases of angiogenesis at various concentrations and over varied amounts of time.

U.S. Pat. No. 6,855,345 B2 provides persons with diabetes the opportunity to safely and conveniently take measures to reduce the likelihood of contracting diabetes, as well as the opportunity to safely treat existing diabetes conditions using a natural formulation and method of treatment. It is another object of this patent to provide a nutraceutical formulation comprising one or more processed Morinda citrifolia products that function as an active ingredient in the nutraceutical formulation. The processed Morinda citrifolia products comprise one of a processed Morinda citrifolia leaf and root hot water extract, a processed Morinda citrifolia leaf and root ethanol extract, a processed Morinda citrifolia leaf and root steam distilled extract, a processed Morinda citrifolia fruit juice or fruit juice concentrate, a processed Morinda citrifolia puree juice or puree juice concentrate, a processed Morinda citrifolia dietary fiber, a processed Morinda citrifolia oil or oil extract and a processed Morinda citrifolia leaf and root extract. The nutraceutical formulation may further comprise other ingredients, such as carrier mediums, water, other fruit juices, etc., and may be in the form of a liquid, a gel, a capsule, a tablet, a concentrate solution, a powder, or any other type of food product. In addition, the processed Morinda citrifolia product may be embodied in a formulation suitable for intravenous injection or systemic release or administration.

European publication number EP1654003 B1 discloses compounds containing an effective amount of Morinda citrifolia fruit juice to inhibit the conversion of mammary cells to tumors. It provides a non-toxic compound having an anti-tumorigenesis effect, wherein the non-toxic compound comprises an effective amount of Morinda citrifolia product selected from Morinda citrifolia fruit juice and methyl sulfonyl methane and water, wherein the Morinda citrifolia fruit juice is present in an amount of 5 percent by weight and wherein the methyl sulfonyl methane is present in an amount of 5 percent by weight.

PCT publication number WO 200245654 discloses the use of a dietary supplement that provides a cancer preventative effect at the initiation stage of carcinogenesis.

Implementation of this disclosure takes place in association with a dietary supplement that is processed from the fruit of the Indian Mulberry plant, scientifically known as Morinda citrifolia L. In one implementation, the dietary supplement includes reconstituted Morinda citrifolia fruit juice from pure juice puree of French Polynesia. The supplement may also include other natural juices, such as a natural grape juice concentrate, a natural blueberry juice concentrate, and/or another natural juice concentrate. In one implementation, the dietary supplement is not processed from dried or powdered Morinda citrifolia, rather liquid is extracted from the fruit of the Morinda citrifolia and used to create the dietary supplement. The dietary supplement disclosed herein reduces the DMBA-DNA adduct formation in various organs, such as the heart, kidneys, lungs, and liver. The DNA adducts formation in various organs, such as the heart, kidneys, lungs, and liver. The DNA adduct formation furthers chemical carcinogenesis. The use of the dietary supplement protects cells and/or lipids from oxidative modification mediated by SAR. As such, the use of the dietary supplement contributes to cancer inhibitation.

U.S. Pat. No. 7,070,813 discloses methods and formulations or compositions for the treatment of colon cancer, and particularly for the inhibition, prevention and/or reduction of cancerous cell growth, as well as the destruction of early stage cancerous cells within the colon region of a mammal, wherein the formulations and compositions comprise an identified amount or concentration of a processed Morinda citrifolia product or an active ingredient there from, as obtained from the Indian Mulberry plant.

WO 2005025410 discloses formulations and methods for treating mammary breast cancer during its initial phases, as well as for preventing mammary breast cancer, by providing a safe, nutraceutical formulation comprising Morinda citrifolia, methylsulfonylmethane (MSM), and other ingredients.

Taking into consideration of the above prior art, anti-leukemia and anti-lung cancer properties of a plant from the family Rubiaceae, Acanthaceae and Zingiberaceae have never been known. Hence, it is advantageous to provide a method and composition comprising a plant extract from the family Rubiaceae, Acanthaceae and Zingiberaceae that is effective against leukemia and lung cancer.

### SUMMARY OF THE INVENTION

The present invention aims to provide compositions and methods for pharmaceutical or nutraceutical use in an animal or human. In addition, methods for manufacturing the compositions are also provided.

The present invention further aims to provide compositions and methods for treating, as well as preventing and inhibiting blood and lung cancer by providing a safe, nutraceutical composition comprising a plant extract from the family Rubiaceae, Acanthaceae and Zingiberaceae. In more specific, the nutraceutical composition is capable of inducing apoptosis in leukemia and lung cancer cells, to reduce severity and incidence, without affecting growth of normal cells.

In a first aspect of the present invention, there is provided a nutraceutical composition comprising an extract from the family Rubiaceae, Acanthaceae and Zingiberaceae, which is effective against blood cancer (or leukemia) and lung cancer cells.

Preferably, the plant from the family Rubiaceae is derived from any vegetative parts (for example leaves, roots, etc.) of any one or combination of Morinda citrifolia, Morinda elliptica, Morinda umbellata, Morinda rigida, Labisia spp., Orthosiphon spp and Eucheuma spp.

Preferably, the plant from the family Acanthaceae is derived from any vegetative parts (for example leaves, roots, etc.) of Clinacanthus spp.

Preferably, the plant from the family Zingiberaceae is derived from any vegetative parts (for example leaves, roots, etc.) of any one or combination of the plant species of Zingiber zerumbet (main bioactive as zerumbone), Zingiber aromaticum, Zingiber cassumunar, Zingiber chrysostachys, Zingiber citrinum, Zingiber gracile, Zingiber griffithii, Zingiber officinale, Zingiber ottensii, Zingiber puberula and Zingiber spectabile.

In a second aspect of the present invention, there is provided use of a composition comprising an extract from the family Rubiaceae, Acanthaceae and Zingiberaceae in the manufacture of a medicament against blood cancer (or leukemia) and lung cancer cells.

In a third aspect of the present invention, there is provided a method to prepare a composition comprising extracts from the family Rubiaceae, Acanthaceae and Zingiberaceae, which is effective against blood cancer (or leukemia) and lung cancer cells.

In accordance with the invention as embodied and broadly described herein, the present invention features a nutraceutical composition for treating, as well as preventing and inhibiting blood and lung cancer comprising processed Morinda citrifolia present in an amount between about 0.001 and 100 percent by weight, Clinacanthus spp. present in an amount between about 0.001 and 100 percent by weight, and Zingiber zerumbet present in an amount between about 0.001 and 100 percent by weight.

In one preferred exemplary embodiment, the nutraceutical composition further comprises methylsulfonylmethane (MSM) present in an amount between about 0.001 and 100 percent by weight.

The composition of the present invention can be formulated and can stand alone as a complete product preparation for treating, as well as preventing and inhibiting blood and lung cancer, or it may be included as a compound ingredient in other products. Non-limiting examples of other products include food, medicine, beverages, meal replacement drinks, dietary supplement capsules, tablets, or a simple water additive.

The formulation can be prepared in a variety of forms. Non-limiting examples of such forms include capsules, tablets, pills, dispersions, suspensions, solutions, powders, teas, syrup concentrates, bars and chews. It can be further packed packages in bottles or other packaging for sale.

In a fourth aspect of the present invention, there is provided a method of treating, as well as preventing and inhibiting blood and lung cancer in a mammal, comprising administering to the mammal in need thereof an effective amount of the composition described herein. The mammal may be a human or an animal, but preferably a human.

In an embodiment, the administration of composition disclosed herein to a mammal is via an oral route.

In another embodiment, the composition is administered to a mammal at a dose of about 0.1 to 5000 mg/kg of body weight of a mammal per day. More preferably, the usual dose or therapeutically effective amount of the composition is in the range from about 5 to 500 mg/kg of body weight of a mammal administered in equal portions twice a day or thrice a day.

These and other features and advantages of the present invention will be set forth or will become more fully apparent in the description that follows and in the appended claims. The features and advantages may be realized and obtained by means of the instruments and combinations particularly pointed out in the appended claims.

Furthermore, the features and advantages of the invention may be learned by the practice of the invention or will be obvious from the description, as set forth hereinafter.

### BRIEF DESCRIPTION OF THE DRAWINGS

The invention description below refers to the accompanying drawings, of which:

FIG. 1 are graphs showing the Reduction of viability measured by MTT test (mean±S.D. % Absorbance; n=3) by each Morinda citrifolia leaf (concentration 0 to 200 μg/ml)) on (a) MRC5 (Human lung fibroblast (MRC5), (b) A549 cells human lung adenocarcinoma (A549) cell lines) and (c) Wehi-3B (leukemia cancer cells) after 72 hours;

FIG. 2 are Normal phase contrast inverted micrographs (microscope photographs) showing morphological changes of A549 cells (lung cancer) treated with (a) control A549 cells; (b) Morinda spp. leaves erlotinib after 24 h: most of the cells exhibit normal morphology while some cells show cell shrinkage; (c) Morinda spp. leaves erlotinib after 48 h: clear apoptogenic morphology such as blebbing and cell shrinkage observed; and (d) Morinda spp. leaves erlotinib after 72 h: most of the cells were detached with obvious cell shrinkage, and blebbing of the cell membrane. BL: blebbing of the cell membrane; CS: cell shrinkage (400× magnification);

FIG. 3 are Fluorescence micrographs of AO/PI double-stained on treated-A549 cells (lung cancer), in which micrograph (a) refers to untreated cells showing normal structure without prominent apoptosis; micrograph (b) refers to Morinda spp. leaves treated with erlotinib after 24 h: cells exhibit blebbing of the cell membrane and bright green nucleus showing condensation of chromatin; micrograph (c) refers to Morinda spp. leaves treated with erlotinib after 48 h; late apoptosis features were seen by representing intercalated AO (bright green) amongst the fragmented DNA; and micrograph (d) shows Morinda spp. leaves treated with erlotinib after 72 hours; more late apoptosis, orange colour represents the hallmark of late apoptosis while red color represents secondary necrosis or dead cells (72). VI: viable cells; BL: blebbing of the cell membrane; CC: chromatin condensation; AB: apoptotic body; LA: late apoptosis; SN: secondary necrosis (200× magnification);

FIG. 4 are microscope photographs showing morphological changes of lung cancer leukemia cells after treatment by (a) Morinda citrifolia leaf and Zerumbone, (b) control cells treated with 0.1% DMSO, and (c) cells treated with 17.0 μg/ml Morinda citrifolia leaf and Zerumbone after 24 hours, 48 hours, 72 hours incubation;

FIGS. 5A, 5B and 5C are three graphs showing the colourimetric assay of caspase 3, 8 and 9 in A549 (lung cancer) treated and untreated with Morinda citrifolia leaf and Zerumbone for 24 hours, 48 hours and 72 hours. Cells were cultured in RPMI1640 (25 ml flask) media maintained at 37° C. and 5% CO₂. Independent t-test showed a significance (*p<0.05) between control and treated cell activity of caspase 3, 8 and 9;

FIG. 6 is a graph showing the colourimetric assay of caspase 3 and 8 in leukemic cells (Wehi-3B) treated and untreated with Morinda citrifolia leaf and Zerumbone for 12 hours, 24 hours, 48 hours. Cells were cultured in RPMI1640 (25 ml flask) media maintained at 37° C. and 5% CO₂. Independent t-test showed a significance (*p<0.05) between control and treated cell activity of caspase 3 and 8;

FIG. 7 shows the Flow cytometric analysis of Annexin V on treated-A549 cells (lung cancer). (a) untreated cells; followed by Morinda spp. leaves after (b) 12 h; (c) 24 h; (d) 48 h; (e) 72 h; and also erlotinib after (f) 12 h; (g) 24 h; (h) 48 h; and (i) 72 h. Data are shown as mean±SD (n=3). * a, b, c mean within time differed significantly across treatment group at p<0.05 as compared with control; w, x, y, z mean within treatment group differed significantly across time at p<0.05 as compared with control;

FIG. 8 shows the flow cytometric analysis of cell cycle distribution of Wehi-3B (blood cancer) cells which were treated with 17.0 μg/ml Morinda citrifolia leaf and Zerumbone after 12 hours, 24 hours, 48 hours, n=3 incubation. Data shown as mean±SEM. *P<0.05 vs control. cells were cultured in RPMI1640 (25 ml flask) media maintained at 37° C. and 5% CO₂;

FIG. 9 shows the flow cytometric analysis of cell cycle phase distribution of treated-A549 cells (lung cancer). (a) Control, (b) Morinda spp. leaves after (b) 12 h; (c) 24 h; (d) 48 h; (e) 72 h; and also erlotinib after (f) 12 h; (g) 24 h; (h) 48 h; and (i) 72 h. Data are shown as mean±SD (n=3). Sub-G0/G1 peak denoting apoptotic cells with hypodiploid DNA content, and follow by G0/G1, S, and G2/M phase. Both Morinda spp. leaves and erlotnib induced G0/G1 arrest in cell cycle progression on A549 cells. * a, b, c mean within time differed significantly across treatment group at p<0.05 compared with control; w, x, y, z mean within treatment group differed significantly across time at p<0.05 compared with control;

FIG. 10 shows results of TUNEL assay of Wehi-3B cells which were treated with 17.0 μg/ml Morinda citrifolia leaf after 72 hours, n=3 incubation. Data shown as mean±SEM. *P<0.05 vs control. cells were cultured in RPMI1640 (25 ml flask) media maintained at 37° C. and 5% CO₂;

FIG. 11 shows the comparative lung tumor weights (mean±SD) between untreated and Morinda spp. treated-mice (p<0.05);

FIG. 12 shows the H&E and IHC staining for EGFR (epithelial growth factor) expression on tumor tissues (Magnification, ×200), (a) Pleomorphic-hyperchromatic cells (black arrow) & poorly differentiated tumor (dashed black arrow), newly formed blood vessels (angiogenesis) (yellow arrow), tumor mass mixed with proliferated collagen connective tissue supporting stroma (red arrow). (b) Variable sized and shaped hyperchromatic nuclei, the tumor cells (yellow arrow), amorphous pinkish pool of proteinicious materials in which the tumor cells floated within (dashed black arrow). Proliferated fibroblastic-like tumor cells are disseminated in a twisted whirlpool-like pattern (black arrow). (c) Immunohistochemistry reaction of extracellular matrix ground substance of a fibroscromas like tumor mass to (EGFR) antibody, golden-brown positive reaction (yellow arrow). Some tumor mass cells positive reaction to EGFR indicted by their deep brown color (black arrow). (d) Immunohistochemical staining with EGFR, of extensive immuno-positive reaction in the stromal supportive tissue of tumor mass indicated by the presence of deep to light golden brown color (black arrow), & deep-brown color (yellow arrow). Tumor cells immuno-negative reaction to EGRF indicated by their counter stained Hematoxylin blue color (dashed black arrow); and

FIG. 13 is a block diagram showing the proposed model of Morinda spp. and Clinacanthus spp. leaves extract mechanism of action for G0/G1 arrest and apoptosis via in vitro and in vivo. FIG. 10 is.

### DETAILED DESCRIPTION

A person skilled in the art will readily appreciate that the present invention is well adapted to carry out the objects and obtain the ends and advantages mentioned, as well as those inherent therein. The embodiment describes herein is not intended as limitations on the scope of the invention.

The term “therapeutically effective amount” used herein throughout the specification refers to the amount of the active ingredient, the extract, to be administered orally to the subject to trigger the desired effect without or causing minimal toxic adverse effect against the subject. One skilled in the art should know that the effective amount can vary from one individual to another due to the external factors and age, sex, diseased state, races, body weight, formulation of the extract, availability of other active ingredients in the formulation and so on.

The present invention provides novel compositions for nutraceutical or pharmaceutical use in a mammal, preferably in a human. In an embodiment, the composition comprises a plant extract from the family Rubiaceae, Acanthaceae and Zingiberaceae at about 0.1% to 100% w/w based on the total weight of the composition.

Preferably, the plant from the family Rubiaceae is derived from any vegetative parts (for example leaves, roots, etc.) of any one or combination of Morinda citrifolia, Morinda elliptica, Morinda umbellata, Morinda rigida, Labisia spp., Orthosiphon spp and Eucheuma spp.

Preferably, the plant from the family Acanthaceae is derived from any vegetative parts (for example leaves, roots, etc.) of Clinacanthus spp.

Preferably, the plant from the family Zingiberaceae is derived from any vegetative parts (for example leaves, roots, etc.) of any one or combination of the plant species of Zingiber zerumbet (main bioactive as zerumbone), Zingiber aromaticum, Zingiber cassumunar, Zingiber chrysostachys, Zingiber citrinum, Zingiber gracile, Zingiber griffithii, Zingiber officinale, Zingiber ottensii, Zingiber puberula and Zingiber spectabile.

Any combination of proportions of the extracts of Rubiaceae, Acanthaceae and Zingiberaceae is envisioned to be encompassed by the compositions disclosed herein. The percentage provided herein refers to the w/w ratio based on the total weight of the extract portion of the composition and does not include any excipient and extra ingredients added to a formulation.

The combination of these extracts may enhance their functions compared to that when administered alone. Therefore, the combination synergizes the activity of the extracts, as well as decreases one or more toxic effects of the constituent. Compositions of the present invention may be, for example, solid, liquid, suspension, concentrate or powder formulations comprising one or more of the extracts as disclosed herein.

Compositions of the present invention may also comprise other components, for example, one or more excipients, catechins, scopoletin, flavonoids and antioxidants derived from plants or food sources.

The composition of the present invention can be formulated and can stand alone as a complete product preparation for treating, as well as preventing and inhibiting blood and lung cancer, or it may be included as a compound ingredient in other products. Non-limiting examples of other products include food, medicine, beverages, meal replacement drinks, powders, dietary supplement capsules, tablets, or a simple water additive.

The formulation can be prepared in a variety of forms. Non-limiting examples of such forms include capsules, tablets, pills, dispersions, suspensions, solutions, powders, teas, syrup concentrates, bars and chews. It can be further packed packages in bottles or other packaging for sale.

The desired compounds to be extracted from the extract of the invention are mainly constituted of, but not limited to, biophenols, proteins, lipids, saccharides, minerals and small peptides.

In a second aspect, the present invention provides use of the composition described herein comprising a plant extract from the family Rubiaceae, Acanthaceae and Zingiberaceae in the manufacture of a preparation for treating, as well as preventing and inhibiting blood and lung cancer in a mammal.

The preparation is preferably a pharmaceutical preparation that can be formulated into therapeutic dosage forms and tablets, capsules, liquid orals, sterile injections, aqueous or oily solutions or suspensions and the like. The preparation may be administered as food ingredients by known techniques, and oral and parenteral administration (including subcutaneous injection, intravenous or intramuscular technique), in dosage unit formulations containing conventional non-toxic pharmaceutically acceptable carriers, diluents or excipient. The extract of the leaves of Rubiaceae, Acanthaceae and Zingiberaceae family, as it is in the preparation, may be a liquid, paste or a solid powder. As used herein, the term pharmaceutically acceptable carrier means a non-toxic, inert solid, semi-solid or liquid filler, diluent, encapsulating material or formulation auxiliary of any type. Some examples of materials that can serve as pharmaceutically acceptable carriers are sugars and lactose, glucose, and sucrose; starches and corn starch and potato starch; cellulose and its derivatives and sodium carboxymethyl cellulose, ethyl cellulose and cellulose acetate; powdered tragacanth; malt; gelatin; talc; excipients and cocoa butter and suppository waxes; oils and peanut oil, cotton seed oil, safflower oil, sesame oil, olive oil, corn oil, and soybean oil; glycols, and propylene glycol; esters, and ethyl oleate and ethyl laurate; agar; buffering agents and magnesium hydroxide and aluminum hydroxide; alginic acid; pyrogen-free water; isotonic saline; Ringer's solution; ethyl water or alcohol; and phosphate buffer solutions, as well as other non-toxic compatible lubricants and sodium laryl sulfate and magnesium stearate, as well as coloring agents, releasing agents, coating agents, sweetening, flavoring and perfuming agents; preservatives and antioxidants can also be present in the composition, according to the judgment of the formulator.

The pharmaceutical and comestible preparation of the present invention may be prepared by mixing the various components of the preparation using conventional methods. The preferred composition of the preparation may be prepared according to the preferred constituent ranges. In an embodiment, the usual dose or therapeutically effective amount of the extract varies from about 0.1 to 5000 mg/kg of body weight of the patient per day. More preferably, the usual dose or therapeutically effective amount of the extract is in the range of from about 5 to 500 mg/kg of body weight of the patient administered in equal portions twice a day or thrice a day.

The comestibles mentioned herein can be any common daily consumed processed food such as bread, noodles, confections, chocolates, beverages (for example instant tea preparation), and the like. One skilled in the art shall appreciate the fact that the aforesaid extract can be incorporated into the processed comestibles, capsules, tablets or topical medicine during the course of processing. Therefore, any modification thereon shall not depart from the scope of the present invention. As set forth in the above description, the pharmaceutical preparation with activities of suppressing blood and lung cancer, as well as killing the said cancer cells in mammals, so that the preparation can be effectively used for the prevention and treatment of leukemia and lung cancer comprises water or alcoholic or solvent extract or their combinations from vegetative parts (e.g. leaves, roots, etc.) of Rubiaceae, Acanthaceae and Zingiberaceae family, or water or alcoholic or solvent mixture extract of vegetative parts (e.g. leaves, roots, etc) of Rubiaceae, Acanthaceae and Zingiberaceae family.

Preferably, the plant from the family Rubiaceae is derived from any vegetative parts (for example leaves, roots, etc.) of any one or combination of Morinda citrifolia, Morinda elliptica, Morinda umbellata, Morinda rigida, together with Elaeis spp., Labisia spp., Orthosiphon spp and Eucheuma spp.

Preferably, the plant from the family Acanthaceae is derived from any vegetative parts (for example leaves, roots, etc.) of Clinacanthus spp.

Preferably, the plant from the family Zingiberaceae is derived from any vegetative parts (for example leaves, roots, etc.) of any one or combination of the plant species of Zingiber zerumbet (main bioactive as zerumbone), Zingiber aromaticum, Zingiber cassumunar, Zingiber chrysostachys, Zingiber citrinum, Zingiber gracile, Zingiber griffithii, Zingiber officinale, Zingiber ottensii, Zingiber puberula and Zingiber spectabile.

According to a preferred embodiment, the extract to be incorporated into the comestibles and medicine can be acquired from any known method not limited only to the foregoing disclosed method.

Following another embodiment, the extract is prepared in a concentrated form, preferably paste or powdery form which enables the extract to be incorporated in various formulations of the comestibles, capsules, tablets or topical products. In line with the preferred embodiment, the extract shall be the plant metabolites which are susceptible to an extraction solvent. The compounds and small peptides with the anti-blood and lung cancer, as well as killing cancer cells enhancing properties are those metabolites in the water or solvent or alcoholic extracts. Therefore, the water or solvent or alcoholic mixture extracts of vegetative parts Rubiaceae, Acanthaceae and Zingiberaceae family is preferably derived from the extraction solvent of water, water or alcohol, acetone, m chloroform, liquid CO₂ and any combination thereof. In view of the prominent property of inhibiting lood and lung cancer by the extracts in a subject, further embodiment of the present invention includes a method comprising the step of administrating orally or topically to the subject an effective amount of a mixture extract derived from the vegetative parts Rubiaceae, Acanthaceae and Zingiberaceae family.

The present invention will now be described in further detail by way of non-limiting examples.

### Example 1

### Preparation of Aqueous Extract of Morinda Spp, Clinacanthus Spp and Zingiber Spp

Extraction of Morinda spp or Clinacanthus spp. polar extract
Cleaned and crushed leaves of Morinda spp. or Clinacanthus spp. were mixed with a polar extraction solvent, for example water or alcohol. The extracts were then analyzed by HPLC.
Extraction of Zingiber spp. polar extract
Cleaned and crushed rhizomes of Zingiber spp. were mixed with a polar extraction solvent, for example water or 100% alcohol. The extracts were then analyzed by HPLC.
Method for the preparation of a mixture comprising extracts of Morinda spp., Zingiber spp. and Clinacanthus spp.
The polar extracts of Morinda spp., Zingiber spp. and Clinacanthus spp. were tested individually or mixed in various ratios within the range of 8:1:1 to 1:8:1 and 1:1:8 and used in the present invention, as described herein.

### Example 2

### Toxicity Effect

The cytoxicity properties of the extract were determined by using MTT assay. MTT dye is a water-soluble yellow dye that is decreased in viable cells to MTT-formazan (3-[4,5-dimethylthiazol-2-yl]-3,5-diphenylformazan) that is an insoluble purple colored product, by the succinate dehydrogenase mitochondrial enzyme. The cells were seeded in 96-well Microplates at a density of 2×10⁵ cells/ml. After 24 hours incubation, the cells were treated with various concentrations of Morinda leaf, or Clinacanthus spp. Leaves and root extract (100, 50, 25, 12.5, 6.25, 3.125, and 1.562 μM) for 24, 48 and 72 h. After 72 hours of incubation, 20 μL of MTT solution (5 mg/ml in PBS) was added in each well and the plates incubated for 4 hours. Then, after removing the media the formazan crystals were dissolved with 100 μL DMSO. Finally the absorbance was determined at 570 and 630 nm in a microplate reader (ECAN, Sunrise™, Männedorf, Switzerland). Cell viability was calculated as the percentage of absorbent compared to control. The 50% inhibitory concentration (IC₅₀) value, defined as the amount of Morinda citrifolia leaf, or Clinacanthus spp. Leaves and root extract that inhibits 50% of cell growth, was calculated from the concentration-response curves. All experiments were carried out in triplicate. Three independent experiments performed in triplicate were used for these calculations.

The following formula was used for calculating of inhibitory rate of cell viability: Growth inhibition=(OD control−OD treated)/OD control×100. The cyto-toxicity of extract on leukemia and lung cancer cells was represented as IC50 values.

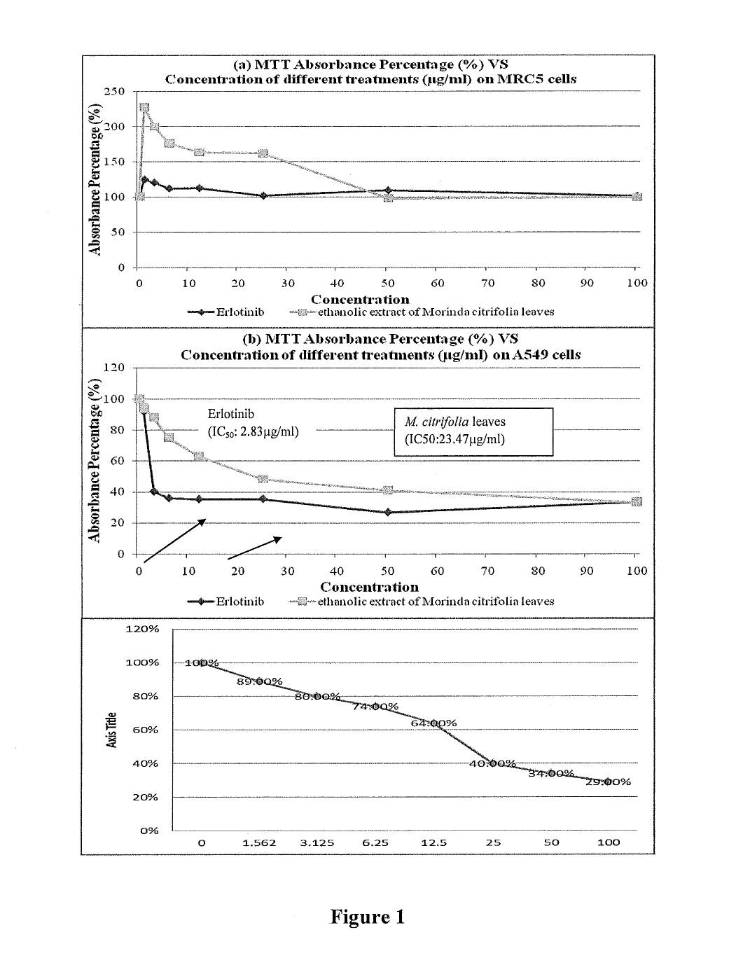

FIG. 1 shows the IC₅₀ concentrations of Morinda spp. Leave extract and erlotinib on A549 cells after 72 hours were 23.47 and 2.83 μg/ml, respectively.

### Example 3

Wehi-3B (blood cancer) or A549 (lung cancer) cells at concentration of 1×10⁶ cells/ml were cultured in RPMI 1640 (ATCC, USA) medium supplemented with 10% fetal bovin serum and were seeded into 25 cm³ culture flask. The next day cells were treated with morinda leaf and root extract at different time periods (12, 24, 48 hours) with IC₅₀ 17.3 μg/ml for Wehi-3B (blood cancer) or A549 (lung cancer) cells. The morphological changes of the cells were observed under normal inverted microscope. Untreated cells were used as negative control. Some morphological appearances such as plasma membrane blebbing, rounding up the cells, cell detachment, cell shrinkage and formation of vacuole in the cell were observed.

Similarly Human lung fibroblast (MRC5), human lung adenocarcinoma (A549) cell lines, Essential Modified Eagle's Medium (EMEM) and Kaighn's Modification of Ham's F-12 (F-12K) medium were obtained from American Type Culture Collection (ATCC). MRC5 and A549 cells were cultured in EMEM and F-12K medium, respectively. Both media supplemented with 10% fetal bovine serum (FBS) and 1% of 100 μg/mL penicillin and streptomycin. Cells were grown in a humidified incubator at 37° C. with 5% CO₂.

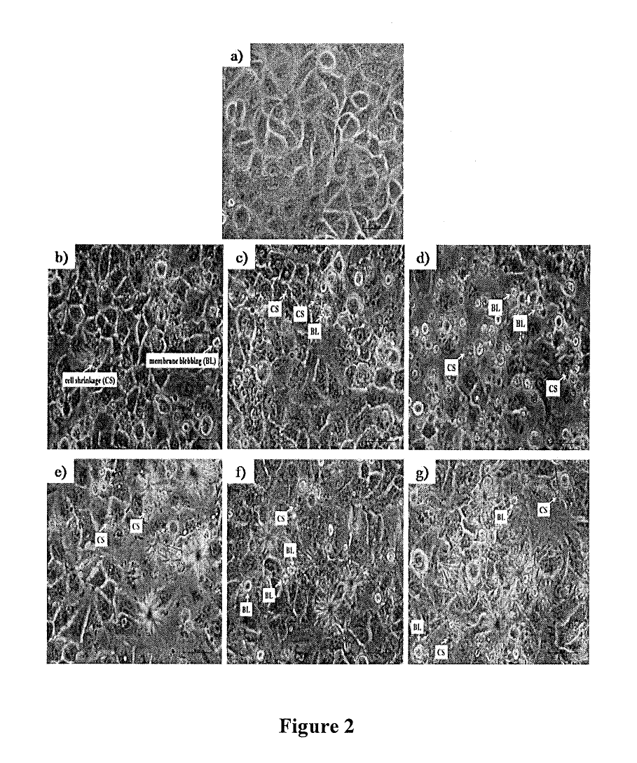

After 24 hours treatment, some cells remained healthy while some cells exhibited cytoplasmic protrusions (FIG. 2(b) and FIG. 2(e)). The morphological changes were distinctively clear in treated A549 cells after 48 and 72 hours treatment with features of prominent growth inhibition, increased blebbing of the cell membrane, shrinkage of cells and extensive nuclear condensation that are the characteristics of apoptosis (FIGS. 2(c), 2(d), 2(f) and 2(g)). In contrast, untreated cells showed typical nonadherent cell morphology and remained healthy and confluent throughout the treatment period (FIG. 2(a)).

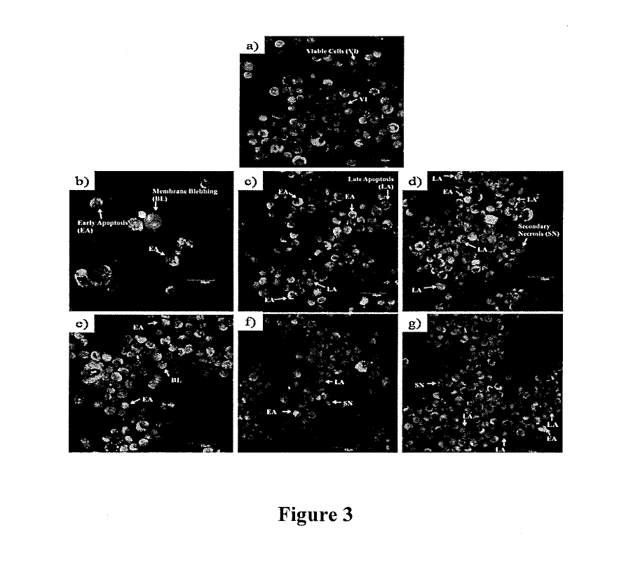

AO/PI double staining was carried out to visualize any morphological changes in the cells upon treatment and also to semi-quantitated viable, apoptotic and necrotic cells, as shown in FIG. 3. Untreated A549 cells showed even distribution of the acridine orange stain as green intact nucleus (denotes healthy cells) with well-preserved morphology (FIG. 3(a)). After 24 hours treatment, blebbing of the cell membrane and dense green nucleus, which indicated nuclear chromatin condensation were noticeable (FIGS. 3(b) and 3(e)). Both early apoptosis features (blebbing and chromatin condensation) and late phases of apoptosis, which specify presence of intense reddish-orange colour due to acridine orange binding to denatured DNA, were observed more apparent at 48 hours of treatment (FIGS. 3(c) and 3(f)) and prominent at 72 hours (FIGS. 3(d) and 3(g)).

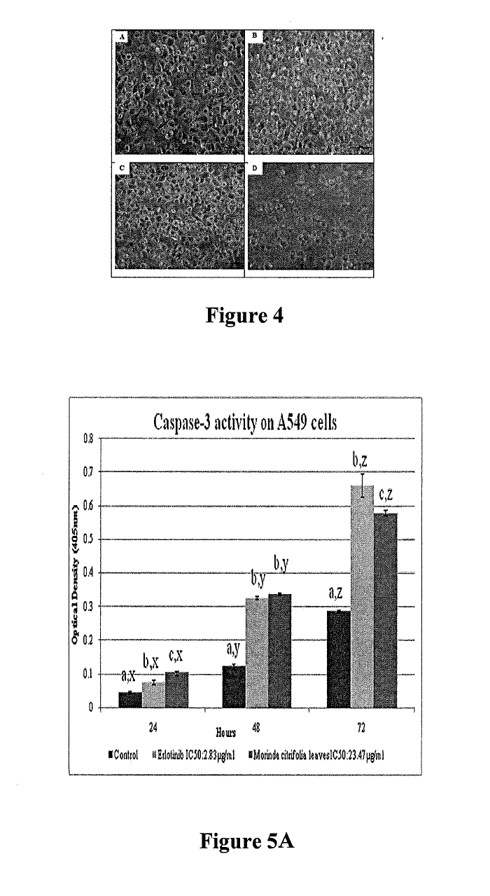

FIG. 4 show similar results with blood cancer cells.

### Example 4

### Colourimetric Assay of Caspase 3 and Caspase 8

The colorimetric assay of caspase 3 was performed using fluorometric and colorimetric assay kit (biotium kit). Wehi-3B (blood cancer) or A549 (lung cancer) cells at concentration of 1×10⁶ cells/ml were cultured in RPMI 1640 (ATCC, USA) medium supplemented with 10% fetal bovin serum and were seeded into 25 cm³ culture flask. The next day cells were treated with morinda leaf and root extract at different time periods (12, 24, 48 hours) with IC₅₀ 17.0 μg/ml. Untreated cells acted as control. The cells were lysed with cold lysis buffer and incubated for 10 minutes on ice. Then, cell lysate was spun down at 10000 rpm for 5 minutes. The supernatant was transferred to new wells and 50 μl assay buffer was added to each samples. Then, 5 μl of enzyme substrate (caspase-3 and 8 substrate) was added to each sample and incubated at 37° c. for 30-60 minutes in a dark place. The samples were read at 495 nm in a dark place. The samples were read at 495 nm in a microplate reader. Finally, the results were shown as optical density (495 nm, mean SD).

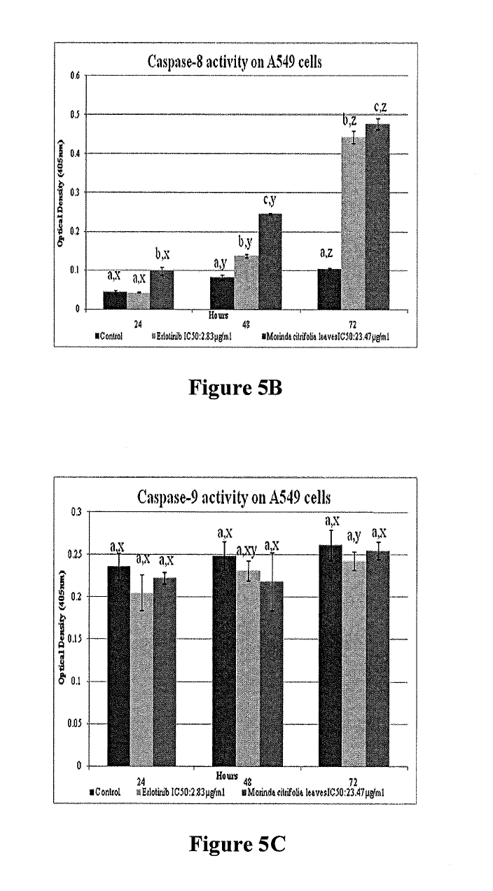

Morinda spp. leaves or Clinacanthus spp. leaves and erlotinib significantly (P<0.05) increased the activities of caspase-3 and -8 (see FIGS. 5A and 5B) in a time dependent manner whilst the activity of caspase 9 (see FIG. 5C) remained unchanged throughout the treatment period as compared with the untreated cells. This data suggested that Morinda spp. leaves induce apoptosis in A549 cells via death receptor pathway.

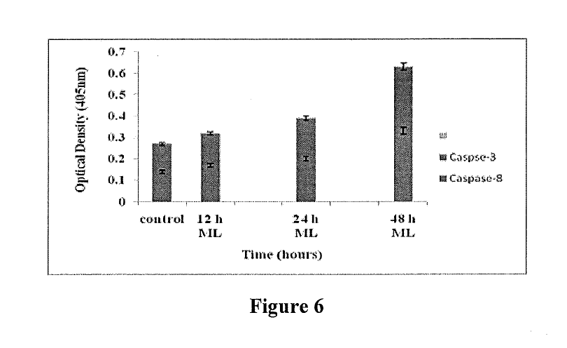

FIG. 6 shows the Caspases-3 and -8 were significantly increased in all Morinda spp. leaves, Clinacanthus spp. leaves and Zingiberaceae rhizome extract combinations as compared to the untreated cells. Results were expressed as the optical density (405 nm) SD of three independent experiments. * a, b, c mean within time differed significantly across treatment group at p<0.05 as compared with control; w, x, y, z mean within treatment group differed significantly across time at p<0.05 as compared with control.

### Example 5

### Annexin V Assay

The annexin V assay gives some information about losing of membrane membrane integrity that is one of the early morphological changes related to apoptotic cell death. Wehi-3B (blood cancer) or A549 (lung cancer) cells at concentration of 1×106 cells/ml were cultured in RPMI 1640 (ATCC, USA) medium supplemented with 10% fetal bovin serum and were seeded into 25 cm³ culture flask. The next day cells were treated with morinda leaf and root extract at different time periods (12, 24, 48 hours) with IC₅₀ 17.0 μg/ml. Then, the annexin V assay acted using the annexin V-FITC apoptosis detection kit. After treatment, the bcells were spun down for 10 minutes at 1000 rpm to discard the media. Cells were washed with 1× binding buffer according to the manufacture. The washed cells were re-suspended in 200 μl of binding buffer. After that, 5 μl of annexine V and 10 μl of propidium iodide were added and tubes incubated in dark place for 15 minutes at room temperature. Flow cytometer analysis was used with BD flowcytometry at 488 nm laser emitting excitation light equipped with an argon laser, and the data analysis was performed by using summit V4.3 software.

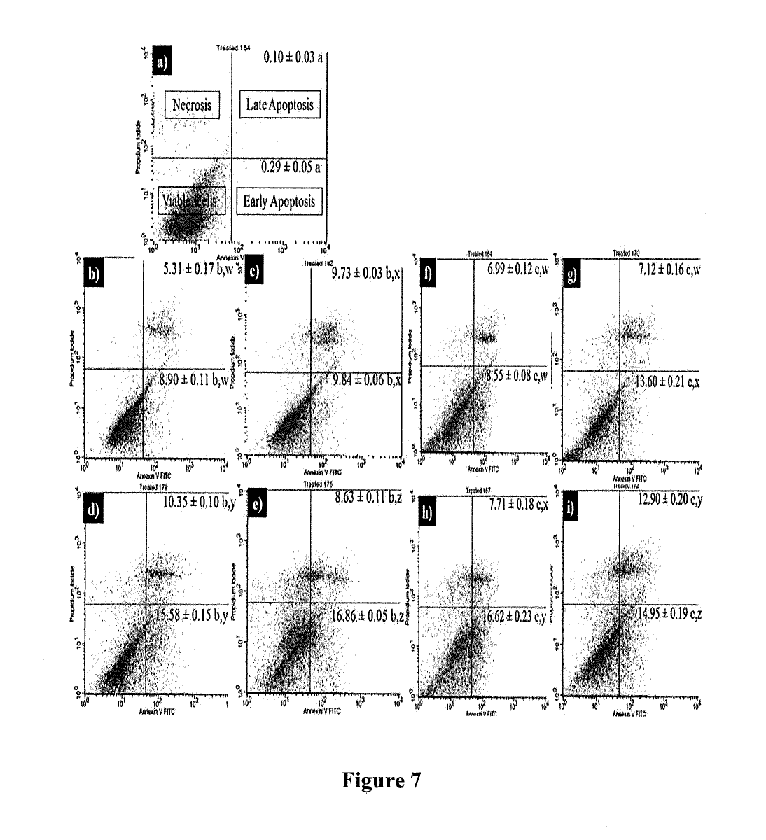

The percentage of apoptotic and necrotic cells after treatment was analyzed after Annexin V-PI staining and the results revealed the percentage of apoptotic cells was significantly higher upon treatment, revealing the cytotoxic mechanism of the extract on A549 cells (FIG. 6). Besides, both Morinda spp. leaves or Clinacanthus spp. leaves and erlotinib induced G0/G1 phase cell cycle arrested in A549 cells (FIG. 7) and accompanied with a concomitant decrease in both S and G2/M phase cells.

TABLE 1
Annexine-V Assay

| | | | | Early |
|---|---|---|---|---|
| | X | Late apoptosis | Normal | apoptosis |
| CONTROL | 0.70 | 1.66 | 96.69 | 0.94 |
| 12 | | | | |
| TREAT 12 H | 0.12 | 8.12 | 80.77 | 10.98 |
| CONTROL | 0.47 | 2.53 | 96.04 | 0.96 |
| 24 H | | | | |
| TREAT | 4.24 | 44.36 | 49.09 | 2.31 |
| 24 H | | | | |
| CONTROL | 0.29 | 2.67 | 92.10 | 4.94 |
| 48 H | | | | |
| TREAT | 4.20 | 42.93 | 51.18 | 1.69 |
| 48 H | | | | |

Table 1 shows flow cytometric analysis of Annexin V of Wehi-3B cells which were treated with 17.0 μg/ml Morinda citrifolia leaf and Zerumbone (X) after 12 hours, 24 hours, 48 hours, n=3 incubation. Data shown as mean±SEM. *P<0.05 vs control. Cells were cultured in RPMI1640 (25 ml flask) media maintained at 37° C. and 5% CO₂.

FIG. 7 shows the Flow cytometric analysis of Annexin V on treated-A549 cells (lung cancer). (a) untreated cells; followed by Morinda spp. leaves after (b) 12 h; (c) 24 h; (d) 48 h; (e) 72 h; and also erlotinib after (f) 12 h; (g) 24 h; (h) 48 h; and (i) 72 h. Data are shown as mean 1 SD (n=3). * a, b, c mean within time differed significantly across treatment group at p<0.05 as compared with control; w, x, y, z mean within treatment group differed significantly across time at p<0.05 as compared with control.

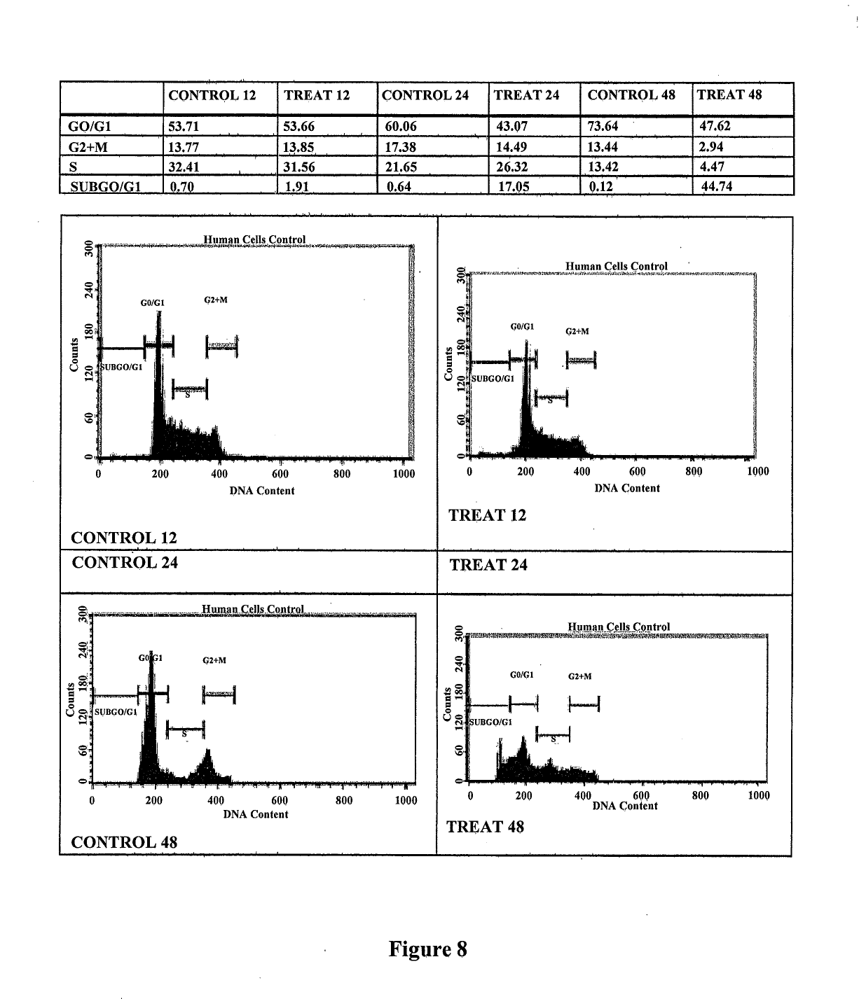

FIG. 8 shows the Flow cytometric analysis of Annexin V on treated-Wehi-3B cells (blood cancer). (a) untreated control cells compared those treated with Morinda spp. leaves and Zingiber extract after 12 hours and 48 hours.

### Example 6

### Cell Cycle

The Wehi-3B (blood cancer) or A549 (lung cancer) cells (2×10⁵ cells/mL) were treated with IC₅₀ concentration of Morinda spp. leaves, or Clinacanthus spp. leaves with or without Zingiber extract and compared with erlotinib. After 12, 24, 48, and 72 hours of incubation, the cells were trypsinized, washed, fixed in 70% ethanol, stained with 1 mg/mL PI and analysed by flow cytometry (BD FACS Canto II, USA) [17].

For flow cytometry analysis fixation of cell population must be performed to keep the integrity and the cell pellets were fixed by adding of 500 μl of 70% chilled ethanol and kept at −20° c. for 5-7 days. Then, the cells were centrifuged at 1000 rpm for 10 minutes and the ethanol was removed. The cells were washed with washing solution containing PBS, EDTA, bovine serum albumin and sodium azide twice, and the pellet cells were resuspended to staining buffer containing 1 ml PBS and 3 μl PI (10 mg/ml) and 10 μl RNase A (50 mg/ml) for 30 minutes on ice dark place. RNase was added in order to permit PI to bind exactly to DNA. The DNA content of cells was analysed with a BD flow cytometer, and the data analysis was performed by using summit V4.3 software.

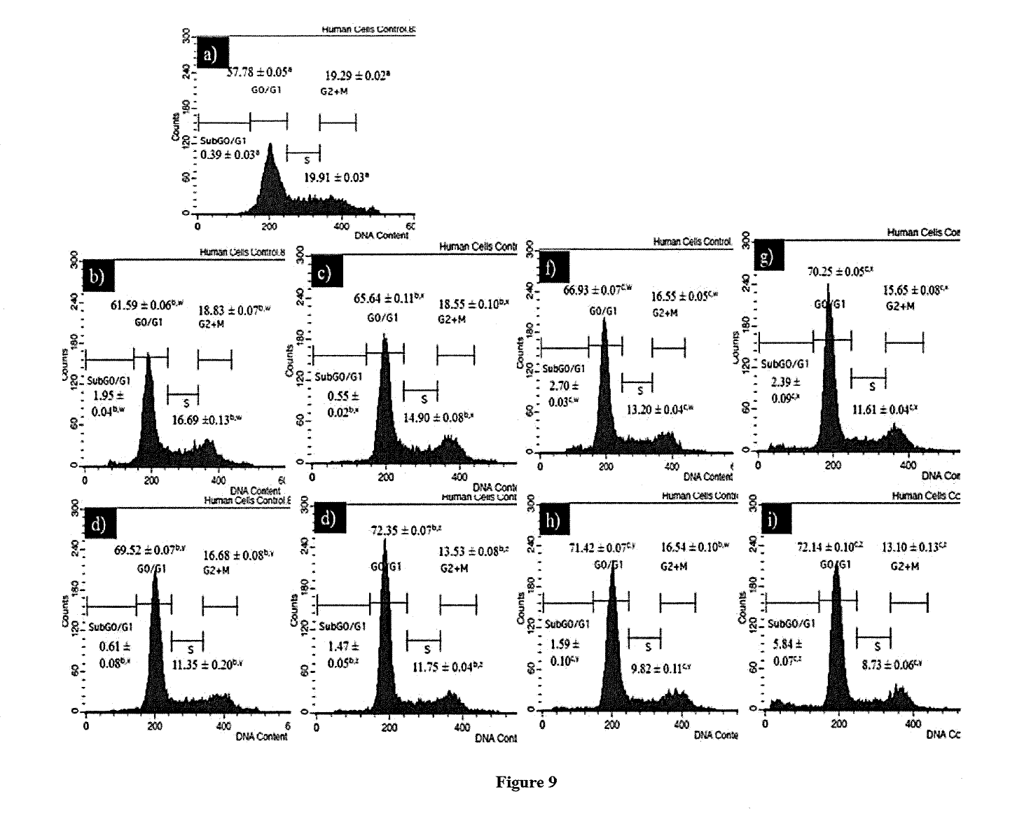

FIG. 9 shows Flow cytometric analysis of cell cycle phase distribution of treated-A549 cells (lung cancer) (a) Control, (b) Morinda spp. leaves after 12 hours; (c) after 24 hours; (d) after 48 hours; (e) after 72 hours; and also erlotinib after (f) 12 hours; (g) 24 hours; (h) 48 hours; and (i) 72 hours. Data are shown as mean±SD (n=3). Sub-G0/G1 peak denoting apoptotic cells with hypodiploid DNA content, and follow by G0/G1, S, and G2/M phase. Both Morinda spp. leaves and erlotnib induced G0/G1 arrest in cell cycle progression on A549 cells. * a, b, c mean within time differed significantly across treatment group at p<0.05 compared with control; w, x, y, z mean within treatment group differed significantly across time at p<0.05 compared with control.

### Example 7

### Tunnel Assay

The induction of apoptosis after incubation of Wehi-3B (blood cancer) or A549 (lung cancer) with Morinda leaf, Clinacanthus leaf and Zingiber root extract (IC₅₀) was evaluated with TUNNEL (Tdt-mediated d UTP Nick-end labelling) assay. 3×10⁶ cells were seeded in 75 cm³ flask and treated with the 17.3 μg/ml for 72 hours. The cells were harvested and centrifuged at 1500 rpm for 5 minutes. The cells were washed twice with PBS and the cells were fixed in 1% paraformaldehyde in PBS and incubated on ice for 15 minutes and were centrifuged at 1500 rpm for 10 minutes and were washed with 5 ml PBS. Then the cells were fixed with 5 ml of 70% (v/v) cold ethanol and were kept in −20° c. for 5-7 days. After this period of time, the cells were spun down at 1500 rpm for 10 minutes and were washed with 5 ml PBS and 1 ml PBS and repeat the centrifugation. Then, 80 μl equilibration buffers were added to each sample and incubated in room temperature for 5 minutes. At the end of incubation, cells were spun down at 1500 rpm for 10 minutes.

The supernatant was removed and 50 μl prepared rTdT (protect from light) containing the equilibration buffer, nucleotide mix and rTdT enzyme, were added to each samples and incubated for 1.5-2 hours at 37° C. in water bath. Then 1 ml EDTA (20 mM) was added to each samples for terminated the RCTN. The cells were centrifuged and removed the supernatant and 1 ml PBS (with 0.1% triton x-100 and 5 mg BSA) was added and the cells were spun down at 1500 rpm for 10 minutes. finally, the staining buffer contain 0.5 ml PBS with 2.5 μg PI and 250 μg RNase A was added to each tubes and were incubated at room temperature in dark place for 30 minutes. Cells were analysed by a BD flow-cytometer where the analysis was performed using summit V4.3 software.

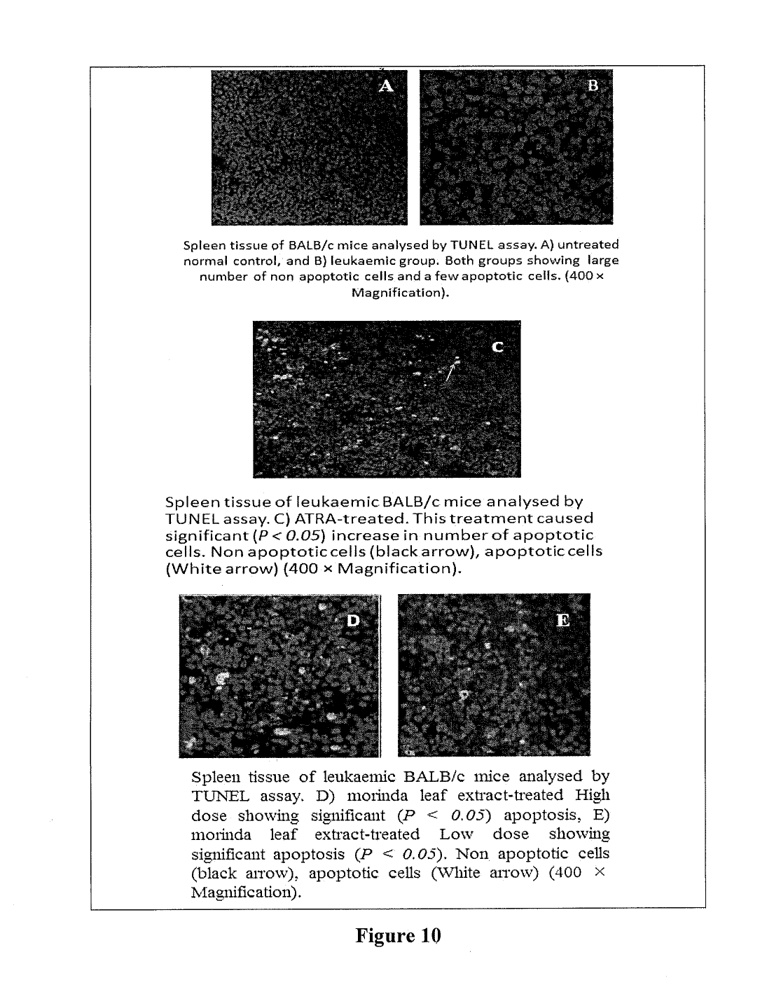

FIG. 10 shows the Spleen tissue analysed by the TUNEL assay. FIG. 10a, which shows untreated normal control and FIG. 10b, which shows leukaemic group of BALB/c mice, contains mostly non apoptotic cells and very few apoptotic cells. However the Spleen tissue of leukaemic BALB/c mice treated with ATRA (see FIG. 10c) or Morinda leaf extract-treated (see FIGS. 10d and 10e) shows significant (P<0.05) apoptosis both at low and high doses used.

### Example 8

### In Vivo Tumor Xenograft Mode

Male BALB/C mice (6 weeks old, weighing 19-20 g) were purchased from Faculty of Veterinary Medicine, University Putra Malaysia. Mice were housed in laminar air-flow cabinets under pathogen-free conditions with a 12-h light/12-h dark schedule and fed with autoclaved standard chow and water ad libitum. A549 cells (2×10⁷) resuspended in 100 μL PBS were injected subcutaneously into the backs of mice. When tumor size reached approximately 100 mm³, after 14 days of implantation, mice were randomly assigned to the following experimental groups and administered daily by oral gavage for 21 days: (a) saline; (b) erlotinib (50 mg/kg) [20]; (c) 150 mg/kg and (d) 300 mg/kg of Morinda spp. leaves, Clinacanthus spp. leaves. Mice were sacrificed via intraperitoneal injection of ketamine HCl (100 mg/kg) and xylazine (10 mg/kg). Tumor was collected. Some were kept in liquid nitrogen for gene expression, while others were fixed in 10% formalin and embedded in paraffin for hematoxylin and eosin (H&E) and immunohistochemical (IHC) examination. IHC kits (ChemMate™ DAKO EnVision™ Detection Kit, Peroxidase/DAB, Rabbit/Mouse) were purchased from Dako, Denmark. The primary antibody was anti-EGFR antibody ab15669 (Abeam; Cambridge, United Kingdom). This protocol was approved by the Animal Care and Use Committee of University Putra Malaysia (UPM/IACUC/AUP-R016/2013).

Data was expressed as mean±standard deviation (mean±SD) of at least three independent experiments; significant differences (p<0.05) using one-way analysis of variance (ANOVA) and Duncan test using statistical analysis IBM SPSS Statistics 21 software.

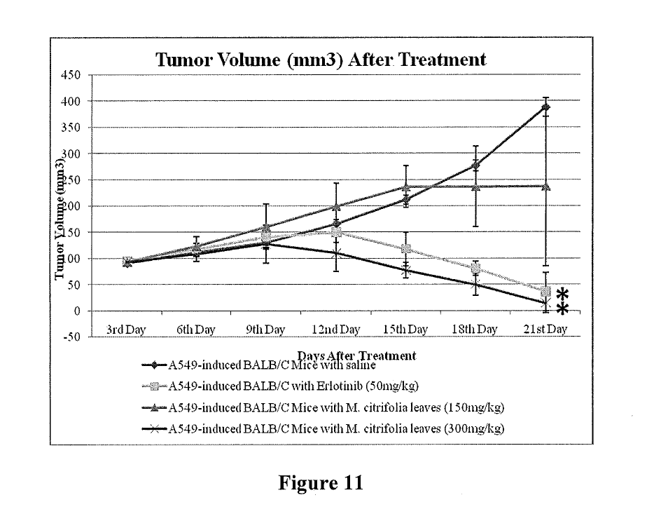

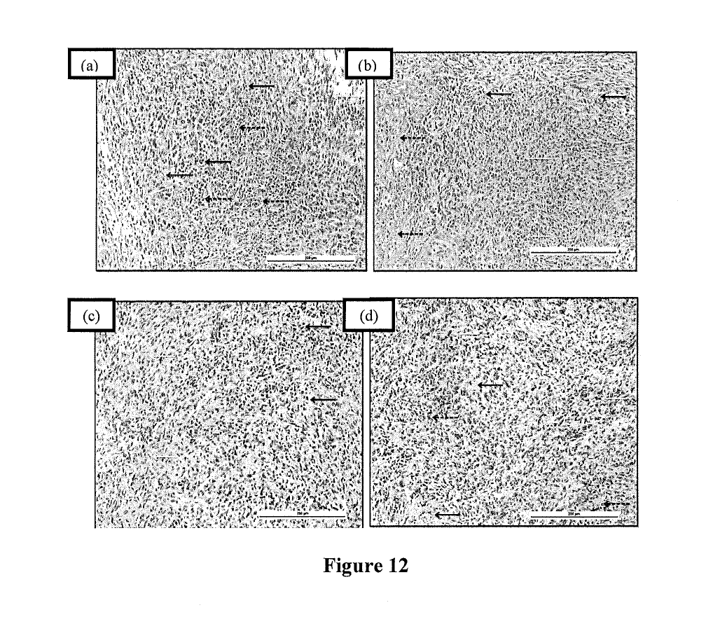

The 300 mg/kg of Morinda spp. leaves or Clinacanthus leaf-treated animals had significantly smaller tumors (36.26±37.00 mm³, 238±152.55 mm³, and 14.81±19.06 mm³ in animals receiving erlotinib, 150 mg/kg and 300 mg/kg of Morinda spp. leaves respectively) (see FIG. 11). H&E staining showed that this xenograft is composed of nodular cell mass, whereas the IHC labeling of tumor sections shows positive EGFR staining (see FIG. 12). Expression of Jak2/Stat3/Stat5a, Bcl-2 and survivin were observed markedly decreased (˜4 fold), while p53 (3.97 fold) was enhanced in 300 mg/kg of Morinda spp. leaves as compared with untreated group (Table 2). However, 150 mg/kg of Morinda spp. leaves was only reported to suppress the Jak2/Stat5a, survivin, and Bcl-2 gene expression as compared with the untreated group. Again, erlorinib acts as positive control indicated down-regulation of survivin and Bcl-2.

FIG. 11 compares the lung tumor weights (mean±SD) between untreated and treated-mice (p<0.05).

FIG. 12 shows the H&E and IHC staining for EGFR expression on tumor tissues (Magnification, ×200). (a) Pleomorphic-hyperchromatic cells with different size and shape can be seen in the given section (black arrow). The cells are oriented in multiple directions with cord-like and cluster-like pattern of growth given the section the so called poorly differentiated tumor (dashed black arrow). Newly foamed blood vessels (angiogenesis) can be seen in the upper right part merging toward the tumor mass (yellow arrow). Massive pinkish amorphous extracellular matrix distributed diffusely within the tumor mass mixed with proliferated collagen connective tissue supporting stroma (red arrow). (b) Variable sized and shaped hyperchromatic nuclei, unidentified cells can be observed within the given section, the tumor cells oriented in different direction and in a variety of forms, like small aggregated clusters or cord like growth (yellow arrow). The tumor cells secreted an abundant of extracellular matrix to facilitate their spread, which appear, here as an amorphous pinkish pool of proteinicious materials in which the tumor cells floated within (dashed black arrow). Proliferated fibroblastic-like tumor cells are disseminated in a twisted whirlpool-like pattern (black arrow). (c) Photomicrograph representing immunohistochemistry reaction of extracellular matrix ground substance of a fibroscromas like tumor mass to (EGFR) antibody, showed the disseminated golden-brown positive reaction to the above-mentioned antibody in tissue section back ground (yellow arrow). Some of the tumor mass cells showed positive reaction to EGFR indicted by their deep brown color (black arrow). (d) Photomicrograph representing immunohistochemical staining with EGFR, revealed extensive immuno-positive reaction in the stromal supportive tissue of tumor mass indicated by the presence of deep to light golden brown color within the section (black arrow). Some of the tumor cells showed immunoposative reaction deep-brown color (yellow arrow). Higher percentage of tumor cells showed immuno-negative reaction to EGRF indicated by their counter stained Hematoxylin blue color (dashed black arrow).

### Example 9

### Gene Expression

RNA was isolated using Trizol (Invitrogen, Carlsbad, Calif.). Custom RT² Profiler PCR Array (CAPM11988), RT² SYBR Green qPCR Mastermix, RT² First Strand Kit and RNase-Free DNase Set were purchased from SuperArray Bioscience Corporation (Frederick, Md.). Quantitative RT-PCR array for differentially expressed genes of interest was performed utilizing RT² Profiler PCR Array Data Analysis version 3.5 (SABiosciences; Fredrick, Md., USA), which normalized to Hsp90ab1 (NM_{—}008302) and Gapdh (NM_{—}008084). RT-PCR data is represented as the average relative mRNA gene expression of each experimental group (n=3).

TABLE 3
Mouse apoptosis RT-PCR-Array on tumor tissues
Quantitative RT-PCR analysis was performed using the comparative
threshold cycle method to calculate fold change in gene expression
normalized to Gapdh and Hsp90ab1 as reference gene. Representative
assays from three independent cohorts each run at least in triplicate
are depicted as the mean fold change from the standardized sample.
Values represent fold differences of individual gene expression between
untreated and treated group. Upper values (>2.00) represent genes
whose expression is up-regulated, lower values (<2.00) genes whose
expression is down-regulated.

| | | | Fold Change | | |
|---|---|---|---|---|---|
| | | | | | Morinda |
| | Accession | | Erlotinib | Clinacanthus | spp. |
| Gene | Number | Description | (50 mg/kg) | leaf | leaves |
| Jak2 | NM_008413 | Janus kinase 2 | −1.26 | −2.12 | −3.42 |
| Stat3 | NM_011486 | Signal transducer and | 1.06 | −1.65 | −4.07 |
| | | activator of transcription 3 | | | |
| Stat5a | NM_011488 | Signal transducer and | 1.42 | −2.59 | −2.82 |
| | | activator of transcription | | | |
| | | 5A | | | |
| Birc5 | NM_009689 | Baculoviral IAP repeat- | −2.40 | −2.93 | −5.89 |
| | | containing 5 | | | |
| Bcl2 | NM_009741 | B-cell leukemia/lymphoma 2 | −3.57 | −2.49 | −4.18 |
| Trp53 | NM_011640 | Transformation related | −1.10 | −1.04 | 3.97 |
| | | protein 53 | | | |

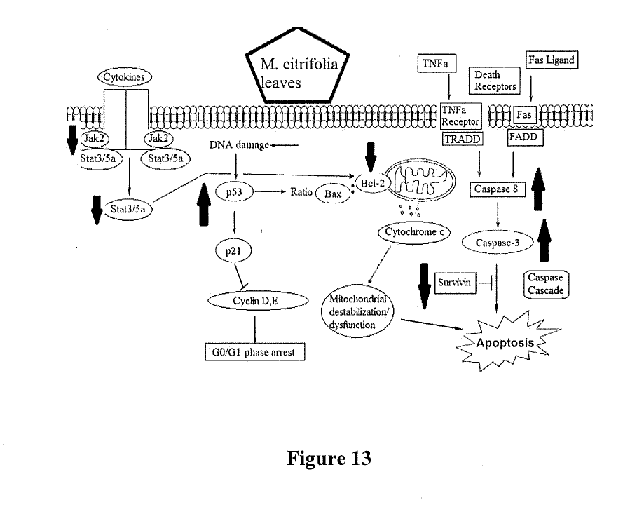

FIG. 13 is the proposed model of Morinda spp. and Clinacanthus spp. leaves extract mechanism of action for G0/G1 arrest and apoptosis via in vitro and in vivo. Morinda spp. leaves and Clinacanthus spp. leaves induced the G0/G1 arrest, apoptosis gene (p53) and extrinsic pathways (caspase-3 and -8) on lung adenocarcinoma, whereas the genes that promoted tumorigenesis (Bcl-2, survivin, Jak2/Stat3/Stat5a) were down-regulated in tumor tissues.

1. A composition having activities of preventing or inhibiting blood and lung cancer progression, wherein the composition comprising an extract from the Rubiaceae, Acanthaceae and/or Zingiberaceae family.

2. A composition according to claim 1, wherein the Rubiaceae family is derived from any one or combination of Morinda citrifolia, Morinda elliptica, Morinda umbellata, Morinda rigida, together with Labisia spp., Elaeis spp, Orthosiphon spp and Eucheuma spp.

3. A composition according to claim 1, wherein the Acanthaceae family is derived from Clinacanthus spp.

4. A composition according to claim 1, wherein the Zingiberaceae family is derived from any one or combination of Zingiber zerumbet, Zingiber aromaticum, Zingiber cassumunar, Zingiber chrysostachys, Zingiber citrinum, Zingiber gracile, Zingiber griffithii, Zingiber officinale, Zingiber ottensii, Zingiber puberula and Zingiber spectabile.

5. A composition according to claim 1, further comprising catechins, scopoletin, phenolic compounds, flavonoids and antioxidants.

6. A composition according to claim 1, wherein the Rubiaceae, Acanthaceae and Zingiberaceae family is in an amount between 0.0001 and 100 percent by weight.

7. Use of a composition comprising an extract from the family Rubiaceae, Acanthaceae and Zingiberaceae in the manufacture of a medicament for preventing or treating blood cancer and lung cancer.

8. The use according to claim 7, wherein the Rubiaceae family is derived from any one or combination of Morinda citrifolia, Morinda elliptica, Morinda umbellata, Morinda rigida, together with Labisia spp., Elaeis spp, Orthosiphon spp and Eucheuma spp.

9. The use according to claim 7, wherein the Acanthaceae family is derived from Clinacanthus spp.

10. The use according to claim 7, wherein the Zingiberaceae family is derived from any one or combination of Zingiber zerumbet (main bioactive as zerumbone), Zingiber aromaticum, Zingiber cassumunar, Zingiber chrysostachys, Zingiber citrinum, Zingiber gracile, Zingiber griffithii, Zingiber officinale, Zingiber ottensii, Zingiber puberula and Zingiber spectabile.

11. The use according to claim 7, further comprising catechins, scopoletin, phenolic compounds, flavonoids and antioxidants.

12. The use according to claim 7, wherein the Rubiaceae, Acanthaceae and Zingiberaceae family is in an amount between 0.0001 and 100 percent by weight.
